(19) United States
(12) Patent Application Publication
(10) Pub. No.: US 2022/0192624 A1
(43) Pub. Date: Jun. 23, 2022

(54) Quantification of contrast-enhanced ultrasound parameteric maps with a radiomics-based analysis

(71) Applicant: The Board of Trustees of the Leland Stanford Junior University, Stanford, CA (US)

(72) Inventors: Dimitre H. Hristov, Stanford, CA (US); Ahmed El Kaffas, San Jose, CA (US)

(21) Appl. No.: 17/606,684

(22) PCT Filed: May 1, 2020
(86) PCT No.: PCT/US2020/031125

§ 371 Date: Oct. 26, 2021

Related U.S. Application Data
(60) Provisional application No. 62/842,269, filed on May 2, 2019.

(51) Int. Cl. A61B 8/06; A61B 8/08; G06T 7/00

(52) U.S. Cl. CPC A61B 8/06; A61B 8/481; G06T 2207/20081; G06T 7/0012; G06T 2207/10132; A61B 8/5207

(57) ABSTRACT

Noninvasive imaging biomarkers to predict cancer treatment response based on early measurements, which would spare non-responding patients from unnecessary side effects and costs of ineffective treatment. Tissue characterization, classification and/or discrimination method is provided to capture different patterns of tissue perfusions. Two or three-dimensional dynamic contrast enhanced ultrasound (DCE US) data of a contrast bolus perfused tissue are acquired or available. Parametric perfusion maps of contrast bolus tissue perfusion parameters representing the DCE US data are generated. For each of the generated parametric perfusion maps statistical parameters are extracted. These statistical parameters, which are based on underlying perfusion characteristics, are first order statistical parameters, second order statistical parameters, or a combination thereof. The method then further classifies and/or discriminates the perfusion maps of the tissue using the extracted statistical parameters.

### FIELD OF THE INVENTION

This invention relates to methods, devices and systems to determine cancer treatment responses using volumetric dynamic contrast-enhanced ultrasound (DCE-US).

### BACKGROUND OF THE INVENTION

Advances in anti-cancer agents have significantly enriched the therapeutic armamentarium available to clinicians for managing disease, but have further complicated patient management because not all patients respond to treatments similarly. There are currently no rapid and efficient methods to determine which treatment regimens will be effective on a patient-by-patient basis at baseline or within weeks of starting treatment.

Conventional anatomical-based assessments with the Response Evaluation Criteria in Solid Tumors (RECIST 1.1) are performed at earliest 2-3 months after treatment start and do not account for acute cytostatic effects that do not always result in anatomical changes in lesion size. Thus, there is a significant need for tools to rapidly assess or predict which patients will respond to treatments, with the potential to spare non-responding patients the high morbidity and cost associated with ineffective treatments.

Histological assessment of tumor vascular properties and perfusion have been demonstrated as predictive surrogates of cancer treatment response, including immunotherapies. Multiple minimally invasive functional perfusion imaging approaches are being explored to predict or monitor response to cancer therapies based on tissue perfusion and/or vascular properties. While many are very promising, radiation exposure (CT/PET), contrast restrictions due to potential adverse events (CT/MRI/PET), limited access (MRI/PET), high cost (MRI/PET), and inability to scan at the bedside are disadvantages for use in repetitive longitudinal exams. Dynamic contrast-enhanced ultrasound (DCE-US) is exempt from these limitations and offers noninvasive bedside functional imaging of perfusion longitudinally.

DCE-US's potential in cancer treatment monitoring applications have been demonstrated. However, conventional use of DCE-US has to date been restricted to 2D ultrasound, with quantified perfusion parameters obtained as averages from a 2D region of interest (ROI) (i.e. conventional ROI-averaged parameters). This renders measurements prone to sampling errors and is unable to consider the heterogeneous nature of tumor perfusion. The recent commercial availability of matrix transducers with contrast-imaging mode has enabled three-dimensional (3D) DCE-US imaging in the clinic as a radiation-free and inexpensive bedside tool for longitudinal imaging to overcome sampling errors attributed to 2D-based imaging. In addition, 3D DCE-US enables volumetric maps of perfusion parameters that can be used to characterize perfusion heterogeneities beyond conventional ROI-averaged parameters.

The use of quantitative histogram and texture features (henceforth image features) has been used to characterize medical images, and more specifically, to capture the heterogeneity of tumor tissue-related parameters in medical imaging and radiomics. It has only been minimally explored for DCE-US in 2D in non-clinical imaging systems. The present invention advances the art by identifying 3D DCE-US perfusion map biomarkers based on repeatable image features sensitive to early treatment-induced changes and correlated to histology, which can then be used to develop models to discriminate between treatment responders and non-responders.

### SUMMARY OF THE INVENTION

The present invention provides a method for quantitative tissue characterization, classification and/or discrimination to capture different patterns of tissue perfusions. In the method two or three-dimensional dynamic contrast enhanced ultrasound (DCE US) data of a contrast bolus perfused tissue are either being acquired or available. Parametric perfusion maps of contrast bolus tissue perfusion parameters representing the DCE US data are generated. For each of the generated parametric perfusion maps statistical parameters are extracted. These statistical parameters, which are based on underlying perfusion characteristics, are first order statistical parameters, second order statistical parameters, or a combination thereof. The method then further classifies and/or discriminates the perfusion maps of the tissue using the extracted statistical parameters.

Statistical parameters are histogram features, texture or radiomic features, or a combination thereof. First order statistics are histogram features and are for example, but not limited to, mean, median, standard deviation, kurtosis, skewness and entropy of grey level values in an image. Second order statistics are texture or radiomic features and are for example, but not limited to, obtained from a co-occurrence matrix (i.e. Grey Level Co-Occurrence Matrix (GLCM), Neighborhood Gray-Tone Difference Matrix (NGTDM), etc.) or similar assessment of voxel inter-relation such as for example, but not limited to, features include energy, homogeneity, correlation texture coarseness, busyness, complexity, among others.

In one embodiment, the parametric perfusion maps represent a temporal behavior of the contrast within a pixel mapped over a two-dimensional space or a three-dimensional space.

In another embodiment, the parametric perfusion maps represent a temporal behavior of the contrast of a single pixel or a window of a group of pixels mapped over a two-dimensional space or a three-dimensional space.

The second order statistical parameters in one example summarize interconnectivity of voxels. In another example, the second order statistical parameters are obtained for different image resolution scales, different imaging pixel or voxel angles, or a combination thereof. In yet another example, the second order statistical parameters are based on the interconnected nature of pixels or voxels in the parametric perfusion maps. In still another example, the second order statistical parameters capture a statistical relationship of one voxel to another voxel with the aim of capturing intensity patterns and heterogeneities with quantified values from different parametric perfusion maps. By focusing on the interconnected nature of voxels, embodiments of the invention are reducing focus on pixel intensities. These intensities are not consistent and affect quantification because of the operator-dependent nature of contrast injection, ultrasound imaging and different physiology. By focusing on the relationship of one voxel to another, the method of this invention places the emphasis on the tissue characterization part, which is more quantitative.

In still another embodiment, the method includes the step of generating multi-parametric features by reducing the dimensionality of the number of correlated statistical features.

In still another embodiment, the method includes using statistical and/or machine learning to characterize tissue and/or classify a disease. For example, supervised machine learning, unsupervised machine learning or deep learning approaches could be used to classify tissues or identify appropriate first or second order features to be used to classify tissues. Such classification examples could differentiate between healthy or diseased tissues based on given clinical standard, or longitudinally assess tissue changes resulting from drug administration.

The method could further include steps of applying an image pre-processing method, a segmentation method, and/or a pixel intensity binning method to enhance the generation of the parametric perfusion maps or the statistical characterization of the parametric perfusion maps.

In an alternate embodiment of the invention, a method is provided in which 3D DCE-US perfusion map biomarkers have been identified based on repeatable image features sensitive to early treatment-induced changes and correlated to histology. This method further provides the use of these features to develop multi-parametric models to discriminate between responders and non-responders and tested on separate preclinical and clinical data. Data demonstrates that early perfusion changes, regardless of treatment or tumor type, predict treatment response.

In yet another alternate embodiment of the invention, a method is provided to determine radiomics-based quantitative features from 3D DCE-US perfusion maps to differentiate between responder and non-responder tumors in pre-clinical and clinical settings. In one example, but not limiting the invention as radiomics can encompass several approaches and models can differ, a multi-parametric linear discriminate analysis (LDA) model was developed using principal components of best features.

Traditional dynamically acquired medical imaging data with contrast signal (where temporal behavior of contrast signal is examined; aka as dynamic contrast enhanced imaging (DCE)) is analyzed by examining a time-intensity curve (TIC) for a single or small group of spatially neighboring voxel, or as an average of voxels within a region of interest. In cases where an ROI is used, the TIC signal doesn't consider the heterogeneity of signal in region. In contrast, on a single or small group voxel basis, the signal doesn't account for the structural information of a single or multiple vessel that may carry the temporal signal, and how a single voxel relates to surrounding voxels. This is because the intensity used in the TIC over time is a first order statistics that accounts only for that one signal as individual not consider how it may relate to other voxels surrounding it. In still another alternate embodiment of the invention, a method is provided that uses second order statistics over time to describe the contrast temporal behavior, as opposed to using a first order statistics approach. In essence, in embodiments of the invention, the signal as a function of surrounding voxels is statistically characterized using measurements such as texture analysis, but obtain over space or over time. This is a unique approach to exam specific characteristics of the contrast agent or the vascular network through which it travels.

One specific application of embodiments of this invention is in contrast ultrasound methods. There are two types of contrasts, targeted and non-targeted. It is important that in targeted contrast enhanced ultrasound, one knows how much of the contrast actually targets or ‘attaches’ to endothelial cells, and to differentiate this targeted signal from non-targeted signals. Using second order statistics over time will help with this by looking at the relationship of one voxel to others nearby and determining if there is flow, or if the signal is ultimately changing the ‘texture’ of the image in manner similar to accumulation of the contrast through targeting. In non-targeted contrast applications, the perfusion in cancers tends to be highly heterogeneous, and thus characterizing it with a single model over a whole ROI is often limited. Other applications in MRI, CT and Ultrasound are also available for quantification of 4D DCE methods.

One advantage of the embodiments of the invention relative to other approaches is that it considers the nature of a signal in a single voxel as a function of surrounding voxels, as opposed to taking that single voxel as an independent system. The invention provides a new way of looking at dynamic signals to provide new quantitative features to be used for perfusion analysis.

### BRIEF DESCRIPTION OF THE DRAWINGS

For interpretation of the gray scale(s) images or data in some of the figures, the reader is referred to Appendix C of the priority document of this application, which is U.S. Provisional Application 62/842,269 filed May 2, 2019.

FIG. 1 shows the method according to an exemplary embodiment of the invention.

FIG. 2 shows a computational pipeline developed to generate parametric maps and extract features PCA and develop the LDA model.

FIG. 3 shows according to an exemplary embodiment of the invention a representative 3D maps of AUC. Noted is heterogeneous perfusion in baseline and longitudinally in treated/control groups.

FIG. 4 shows according to an exemplary embodiment of the invention feature clustergram, ROC and correlation to histology. Specifically, shown is a statistics-based feature selection process based on repeatability and sensitivity to treatment.

FIG. 5 shows according to an exemplary embodiment of the invention feature clustergram, ROC and correlation to histology. Specifically, shown is a heatmap showing all features (y-axis) on days 1, 3, 7 and 10 (D01-D10)—features are measured as percent change from baseline. Left is responder (LS174T) group and right is non-responder (CT26) group, with both treated (T) and control (C).

FIG. 6 shows according to an exemplary embodiment of the invention feature clustergram, ROC and correlation to histology. Specifically, shown is the same as in FIG. 7, but after feature selection. Note that within the responder group, there is an oscillation between treated (T) and control (C) animals from day 1 onwards, not observed in non-responder group.

FIG. 7 shows according to an exemplary embodiment of the invention feature clustergram, ROC and correlation to histology. Specifically, shown is a ROC for conventional parameters alone (PE, AUC, TP, MTT—thin lines), combined in an LDA model (ROI-LDA; 710) and top 2 components from PCA in an LDA model (PCA-LDA2; 720), in the training and test data set.

FIG. 8 shows according to an exemplary embodiment of the invention feature clustergram, ROC and correlation to histology. Specifically, shown is correlations to histology of conventional parameters (top), LDA scores from conventional parameters (ROI-LDA; top), the first component from the PCA (bottom), and the LDA scores from top 16 PCA components (PCA-LDA1) and top 2 PCA components (PCA-LDA2) (bottom). These are shown as absolute measurements, and not percent change from baseline.

FIG. 9 shows according to an exemplary embodiment of the invention representative results in patient 3D DCE-US data. Representative 3D volumetric rendering of contrast signal (A), and cross-section AUC parametric maps from responder and non-responder patients before and within 2 weeks after treatment (B). A) 3D rendering of the AUC parametric map in the volume of interest (VOI). B) Middle cross section of the VOI in the same order for both patients. Top row images are for patient 6 in table 3 (Male, 52 y.o., responder), bottom row images are for patient 4 in table 4 (Male, 68 y.o., non-responder). No significant changes in parametric map appearance is noted in non-responder.

### DETAILED DESCRIPTION

### Experimental Design

In an exemplary and illustrative embodiment, experiments were designed to extract image features from 3D-DCE US longitudinal perfusion maps from liver metastases and to combine these as multi-parametric biomarkers that can differentiate responders from non-responders. To identify reliable features from parametric maps, repeatable histogram and texture features (image features) were isolated to discriminate between responsive and non-responsive tumors treated with the anti-angiogenic agent Bevacizumab on a subject-by-subject basis.

As a proof of concept, the use of two approaches was investigated to generate multi-parametric biomarkers for treatment assessment; i) a statistical approach, and ii) a GLMNET approach, developed on pre-clinical data and tested on pre-clinical test and human test data. Pre-clinical test data included Bevacizumab-treated and control animals, as well as a cohort for feature repeatability assessment. In addition, tested in pre-clinical tissues was the question whether multi-parametric biomarkers were correlated to volumetric histological quantification of vascular densities.

Human data was obtained from ongoing larger trials to assess the feasibility of 3D DCE-US to monitor cancer therapy in patients with liver metastasis from the gastrointestinal (GI) tract; the data was used as initial pilot translational validation of 3D DCE-US parametric map-based biomarkers. Overall, all available subjects (human and pre-clinical) were analyzed, applying stringent inclusion and exclusion criteria to homogenize our study cohorts as was most scientifically reasonable. Clinical data was analyzed with models developed by the inventors from pre-clinical data in a blinded fashion.

### Pre-Clinical Data Groups and Measurements

For the exemplary embodiment, mice were implanted with 20 tumors sensitive to Bevacizumab (LS174T human colon cancer; 10 control and 10 treated, and 20 tumors non-sensitive to Bevacizumab (CT26 murine colon cancer; 10 control and 10 treated were imaged with 3D DCE-US on days 0, 1, 3, 7 and 10 following the start of a Bevacizumab treatment regimen (10 mg/kg on days 0, 3 and 7). Another 20 mice implanted with the LS174T cell line (responsive to treatment) were imaged twice within one scan session to assess repeatability of quantitative parameters. Finally, a total of 18 mice with LS174T tumors responsive to treatment were imaged at 24 hours to further test our biomarkers in a separate cohort of animals; 11 of these animals (5 treated and 6 control) had volumetric histology of CD31 mean vascular density (MVD) quantification to correlate to biomarkers. For all animals, imaging was carried out in 3D using a Philips EPIQ7 ultrasound machine coupled to a clinical X6-1 3D transducer using clinical grade contrast microbubble agents (Definity, Lantheus Medical Imaging, MA, USA) administered using the bolus DCE-US method. For the 20 LS174T animals in the repeatability group, two consecutive bolus data acquisitions were obtained during the same scan session, within 20 minutes of each other, to assess repeatability using the ICC. Additional details on the tumor model and image acquisition are provided in the supplementary methods infra.

### Conventional Bolus DCE-US Perfusion Parameter Extraction

All 3D DCE-US imaging datasets were analyzed using custom Python-based software (see Supplementary Methods infra). A Volume of Interest (VOI) was manually contoured covering the whole tumor visualized on sagittal, longitudinal, and coronal planes in ITKsnap. A first-pass kinetics analysis of the average signal TIC from the VOI after bolus injection was used for the quantification of conventional tumor perfusion parameters from bolus 3D DCE-US. Parameters included the PE and AUC, which are generally related to blood volume, and the TP and MTT, which are estimates of perfusion rates. Perfusion parameters were normalized to the baseline values (pre-treatment scan) to show percent changes.

### Parametric Map Generation

For all 3D DCE-US, a Python pipeline was developed to process images consistently in the same way to extract the bolus-based perfusion parameters on a voxel-by-voxel basis within the same pre-selected VOI used in conventional perfusion analysis. A total of 8 perfusion maps based on tracer model parameters and intensity projections were generated in 3D and saved as NIFTI files. Python-based software was used to generate perfusion maps using parallel processing on a high-performance multi-core processing computing cluster. It has 127-shared servers, which include 16 CPU cores per node, each operating with up to 4 GB of RAM. For parametric maps of tracer models (tens of millions of voxels present in each 3D perfusion image), the software ran in parallel on a single node for each data set, using all 16 cores per node (multi-processing) to parallelize voxel-by-voxel nonlinear least-square fitting of the lognormal model. Maps took an average of 3 hours to be generated. From this process, we obtained five 3D parametric maps and three 3D intensity projections. Detailed processing steps and pipelines are discussed in the supplementary methods. A total of 1128 features were obtained on each day, for each animal.

### Histogram and Texture Feature Extraction

As proof-of-principal of model-based multi-parametric perfusion detection as a surrogate of treatment response, two different linear modeling approaches were evaluated.

Statistical Approach—To reduce the number of features, features were selected that were: i) repeatable using the intra-class correlation coefficient (ICC>0.8) using the repeatability data set, ii) that could significantly differentiate between the treated and control groups in mice with responding tumors at each imaging time point using a rank-sum test with a threshold of p<0.05, and iii) that did not differentiate between the treated and control animals in the treatment-resistant group, with a threshold of p>0.1. Following feature selection, principle component analysis (PCA) was used to isolate representative dimensions of selected features related to control/treated animals, eliminating redundancies (i.e. correlated features) and minimizing dimensionality in the feature set. An LDA model with 10-fold cross-validation was constructed based on the dominant dimensions, as per results.

GLMNET Approach—A GLMNET machine learning model was used to classify the data into responding or nonresponding groups. There was a total of 373 features. All features were evaluated as a relative change from baseline on each longitudinal scan day. Normalization across datasets was performed to account for widespread variations. Using ten-fold cross validation (CV), regularization parameter lambda was selected to minimize over-fitting and CV error for alpha values of 0.3, 0.5, 0.7, where alpha is the balance between L1 and L2 regularization. Features were selected by taking features with nonzero coefficients, and a ROC curve was computed for each alpha value, as well as the corresponding AUC.

### Patient Data

Patient data was obtained from a prospective clinical research study, which consented for 3D DCE-US imaging. Inclusion and exclusion criteria are presented in supplementary methods i. For this purpose, seven adults were scanned, with two scans per patient within two weeks; the first scan right before treatment start and the second scan within 14 days±5 days of treatment start.

### Statistical Analysis and Feature Sorting

Statistical tests were used to evaluate repeatability and significance between different groups as indicated. To test for repeatability, the intra-class correlation coefficient (ICC) was used, where log-transformation was applied to normally distribute data for standard statistical analysis. The 95% confidence intervals (CI) were also calculated for each ICC. Here, an ICC of 0-0.20 indicates no agreement; ICC of 0.21-0.40, poor agreement; ICC of 0.41-0.60, moderate agreement; ICC of 0.61-0.80, good agreement; and ICC greater than 0.80, excellent agreement. An unpaired Wilcoxon rank-sum test was used to compare the statistical significance of different groups with p<0.05 indicating significance. Features were compared in different groups as absolute values, as well as relative changes (see Supplementary Methods infra). A sample size of 10 animals per group was chosen based on an estimation that it would provide 90% power with two-sided 5% error to detect differences as small as 1.5 standard deviations.

Supplementary Methods

### Colon Cancer Xenograft Model of Responder and Non-Responder: Description, Experimentation and Validation

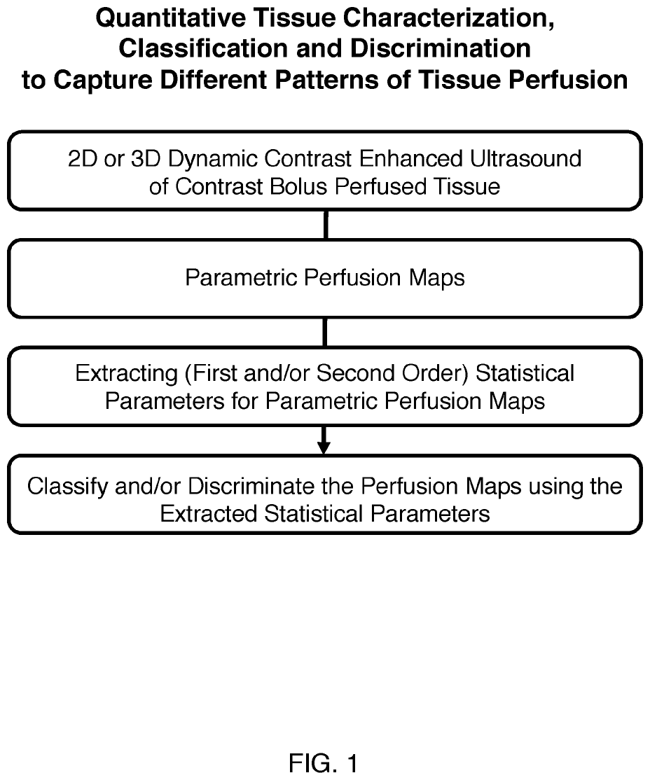

All experimental procedures involving laboratory animals were approved by the Institutional Administrative Panel on Laboratory Animal Care. A total of 78 female nude mice (Charles River, Wilmington, Mass.; 6-8 weeks old; 20-25 g) were induced with human (LS174T; ATCC, Manassas, Va.; n=58) or murine (CT26; ATCC, Manassas, Va.; n=20) colon cancer. The human colon tumor line actively responds to clinical (humanized) VEGF-targeting therapy such as Bevacizumab, while the murine line is resistant to such treatments. Together, these two cell lines can be used to simulate clinical responders and non-responders to anti-angiogenic therapy, with the CT26 cell line acting as a negative control. From the n=58 mice bearing LS174T tumors, 20 were used for control (n=10) and treatment (n=10), 20 were used for repeatability assessment and 18 were used as a separate test data set imaged only at baseline and 24 hours. Of the 18 animals used as test data, 11 had histology at 24 hours for correlation of perfusion features with CD31. Animals bearing CT26 were used as control (n=10) and treatment (n=10) non-responders (negative-control) to Bevacizumab. A complete schematic of the experimental design and animal genotypes is shown in FIG. 1A of Appendix C of U.S. Provisional Application 62/842,269 filed May 2, 2019, which this application claims the benefit from and which is incorporated in its entirety in this application. Both cell lines were grown in Dulbecco's Modified Eagle Medium (DMEM; Gibco, Grand Island, N.Y.), supplemented with 10% fetal bovine serum (Gibco), penicillin (50 U/ml), and streptomycin (50 μg/ml) at 37° C. in a humidified 5% CO2 atmosphere. At 70%-80% confluence, the tumor cells were collected following trypsinization, and 4×10⁶ LS174T cells or 1×10⁶ CT26 cells suspended in 50 μl phosphate-buffered saline and 50 μl Matrigel (BD Biosciences, San Jose, Calif.) were injected subcutaneously on the right lower hind limb of the nude mice. Both LS174T and CT26 tumors were allowed to grow for 10 days to an average diameter of 10 mm (range: 6-14 mm) in the maximum direction (measured with electronic caliper). Treatment responses were previously confirmed using tumor growth and immunohistology. For histological analysis of tumor vasculature, tumors were surgically removed and fixed in 4% paraformaldehyde and PBS solution for 24 h, sectioned into 10 μm slices for CD31 immunofluorescence staining using standard methods. A subset of tumors had several sections cut as previously described to sample volumetric vascular density for correlation. Fluorescent microscopy was performed by using a LSM510 metaconfocal microscope (Zeiss, Maple Grove, Minn.) and a high-resolution digital camera (AxioCam MRc, Bernried, Germany) under 200-fold magnification. The mean vascular density (MVD) per slice was quantified by using Image J software (National Institutes of Health, Bethesda, Md.) as the average value from 5 randomly selected fields of view (single field of view area, 0.19 mm²). The MVD was significantly (p<0.001) lower in bevacizumab-treated compared to control responder tumors (LS174T) [REF]. No significant differences (p>0.05) in the MVD between treated and control tumors were noted in non-responders (CT26). Tumor growth was monitored by using electronic calipers measurements available on the ultrasound system using the following formula: volume=π/6×L×W×H, where L is length, W is width, and H is height. Measurements were significantly different between control and treated responders (LS174T) on day 3, 7 and 10 (p<0.001), but not on day 1 or on any day in non-responders (CT26) (p>0.05).

### Antiangiogenic Treatment

LS174T-bearing mice were randomized into four groups: 1) repeatability group (n=20), 2) treatment group (n=16) or 3) control group (n=15). Treated animals received the anti-angiogenic Bevacizumab (Avastin, Genentech, South San Francisco, Calif.) at baseline (immediately after imaging) as well as on days 3 and 7 (in animals followed beyond 24 hours) after the first injection of the drug. The agent was diluted in sterile saline to a dose of 10 mg/kg b.w. (corresponding to a fluid volume of 10 μl/g b.w.) and administered intravenously. The same volume of saline alone was administered on the same days in control animals. A total of 11 animals in the treatment (n=6) and control (n=5) groups that were imaged and sacrificed at baseline and 24 hours following the start of therapy only, with the goal of characterizing early manifestations of vascular density.

### Three-Dimensional DCE-US Data Acquisition

DCE-US used FDA-approved intravascular ultrasound contrast agents, which has highly echogenic, micron-sized gas bubbles (microbubbles), stabilized by a shell made from biodegradable materials. These enable qualitative tissue perfusion assessment, as well as quantitative parameterization of tissue blood flow and blood volume.

Imaging was carried out in 3D using a Philips EPIQ7 coupled to a clinical X6-1 3D transducer using clinical grade contrast microbubble agents (Definity, Lantheus Medical Imaging, MA, USA) administered using the bolus DCE-US method. Definity microbubbles (FDA-approved perfluoro microbubbles) were diluted 1:4 in sterile 0.9% saline after activation as per manufacturer. A suspension of 120 μL was administered over 5 seconds (at a constant injection rate of 24 μl/sec) with an infusion pump. For the 20 LS174T animals in the repeatability group, two consecutive bolus data acquisitions were obtained during the same scan session. The second bolus was administered about 15 minutes after the first bolus to ensure that the contrast was cleared from the systemic flow of the animal to eliminate signal interference with the second bolus injection signal. For both LS174T and CT26 animals, control and treated tumors were imaged at baseline and at 1, 3, 7, and 10 days following the start of therapy. For each bolus, 3D DCE-US datasets were acquired in real time over four minutes in each mouse by using a built-in Digital Navigation Link of the ultrasound system to custom in-house MevisLab modules written in C++.

For all imaging procedures, mice were anesthetized with inhalation of 2% isoflurane in room air (administered at 2 L/min). Mice were placed prone on a heated imaging stage to maintain constant body temperature for the entire duration of the experiments. A 27G needle catheter (Vevo Micromarker; VisualSonics, Toronto, Canada), which attached to an infusion pump (Kent Scientific, Torrington, Conn.), was placed into one of the two tail veins for contrast agent injection. The transducer was held in a fixed position with a clamp to minimize motion artifacts. To reduce artifacts in the near-field zone of the clinical transducer, a customized standoff ultrasound gel was placed on the skin of all mice. The distance between the transducer and the center of the tumor was set at 3 cm. The following imaging parameters were kept constant for all 3D DCE-US imaging experiments in all tumors: center frequency, 3.2 MHz; mechanical index, 0.09; volume rate, 1 Hz; dynamic range, 52 dB; focus placed beneath the tumor (4-6 cm from transducer head).

### Conventional Bolus DCE-US Perfusion Parameter Extraction

All 3D DCE-US imaging datasets were analyzed using custom Python-based software (available at https://github.com/aelkaffas/3DDCEUSParametricMaps). To generate volumes of interest (VOI), the whole tumor visualized on sagittal, longitudinal, and coronal was manually contoured using ITKsnap to produce volumes of interest.

Image analysis was performed by one reader with 6 years of DCE-US experience in random order. The reader was blinded to the treatment information and types of animals (LS174T vs. CT26). All 3D DCE-US imaging datasets were analyzed with a custom software in MeVisLab. A first-pass kinetics analysis of the signal intensity-time curve from the VOI, which is based on the wash-in and washout kinetics of microbubbles after bolus injection, was used for the quantification of tumor perfusion. For conventional parameter extraction, the average of the linearized intensities of all the pixels in the VOI was used to obtain a time-intensity curve. A lognormal model was fitted to each perfusion curve to extract the following perfusion parameters: peak enhancement (PE, arbitrary units, au), area under the curve (AUC, au), mean transit time (MTT, seconds) and time to peak (TP, seconds). The PE and AUC are generally related to blood volume while the TP and MTT are estimates of perfusion rates. Perfusion parameters on days 1, 3, 7 and 10 were normalized to the baseline values to show percent changes.

### Parametric Map Generation

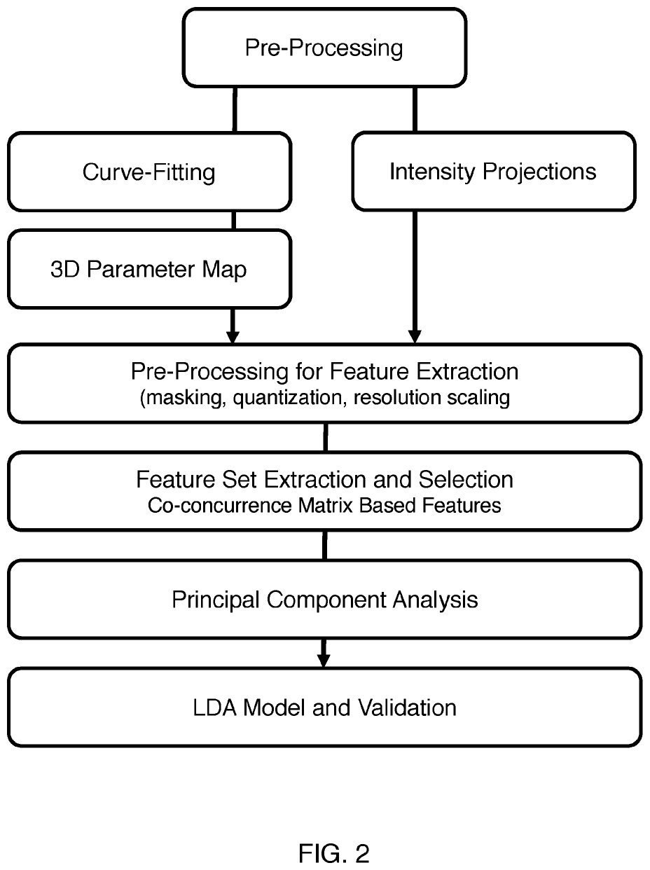

A total of 8 perfusion maps based on tracer model parameters and intensity projections were generated in 3D and saved as NIFTI files. Python-based software was used to generate perfusion maps using parallel processing on a high-performance multi-core processing computing cluster. The cluster is used for voxel-by-voxel least-square fitting of time-intensity curves to an established perfusion model for parametric map generation. It has 127-shared servers, which include 16 CPU cores per node, each operating with up to 4 GB of RAM. For parametric maps of tracer models (tens of millions of voxels present in each 3D perfusion image), the software ran in parallel on a single node for each data set, using all 16 cores per node to perform a voxel-by-voxel nonlinear least-square fitting of the lognormal model. Maps take an average of 3 hours to be generated. Unlike CT and MRI data, ultrasound images are noisy and filed with artifacts. To account for this, several ‘cleanup’ steps were applied to subtract major artifacts in images in the 4D sequence, and to smoothen the time-intensity curves. More specifically, the processing pipeline included: (1) linearization of the signal and isotropic resampling to generate a time intensity curve (TIC); (2) identification of contrast arrival in image (wash-in start) and auto-masking of flow-only voxels (perfused tissue); (3) standardizing TIC fitting (which also checks for quality of fit) to each voxel based on the log-normal perfusion model or an intensity projection; and (4) generation of parametric maps or intensity projection (FIG. 2). Parametric maps include: peak enhancement (PE), area under the curve (AUC), time-to-peak (TP), mean transit time (MTT), arrival time of contrast (TO), maximum intensity projection (MIP), average intensity projection (AIP), and standard deviation projection (SIP). For clinical data, an additional motion correction step was employed. This is necessary given increased motion in clinical data compared to pre-clinical data.

### Histogram and Texture Feature Extraction

Radiomics-based image features can generally be obtained from any image, and can broadly divided into low-level (i.e. histogram) and high-level (i.e. non-texture and texture) features. Low-level features are based on pixel statistics, from histogram intensity-based parameters (mean, median, and skewness of grey levels). Non-texture features are based on segmented lesions such as tumor size/volume (longest diameter, short axis, etc.) and tumor shape (circularity, compactness, etc.). While histogram features capture information regarding the spread and shape of image histograms (such as mean, median, and skewness of grey levels, etc.), texture features consider the overall statistical relationship of one voxel to another with the aim of capturing intensity patterns and heterogeneities with a single quantified value.

This goes beyond basic averages of voxel intensities, and is especially advantageous to focus the quantification on the patterns of perfusion, as opposed to intensities of contrast, which may be affected by attenuation or contrast preparation or administration. These have been shown to be good descriptors of the heterogeneity of tumor tissue or perfusion, and sensitive indicators of treatment response in various imaging modalities.

Histogram and texture features were extracted from each perfusion and intensity projection map from 3D DCE-US data, for each animal or patient data on each scan day. For histogram features, the mean, median, mode, standard deviation, skewness and kurtosis of the intensity distribution were extracted. For texture features, all parametric maps were quantified exactly the same by: i) confining the ROI to the smallest box/region possible in image to speed up the computation, ii) normalization, iii) isotropic resampling of pixels, iv) quantizing of pixels (lloyd quantization) (FIG. 1). Features were extracted at the following 3 length scales by resampling the isotropic voxel size to the following dimension: 0.3, 0.6 and 0.9 mm, in all possible directions. Four different categories of second-order texture features were extracted as described in Table 1. These include the Grey Level Co-Occurrence Matrix, Grey Level Size-Zone Metric, Grey Level Run-Length Matrix and Neighborhood Grey Tone Difference Matrix.

For each animal and at each time point, a total of 1128 features were extracted over all three length scales and all parametric maps/intensity projections. All features and conventional parameters were evaluated as relative change

((X_{D01}−X_{D00})/X_{D00};(X_{D03}−X_{D00})/X_{D00};X_{D07}−X_{D00})/X_{D00};X_{D10}−X_{D00})/X_{D00}),

except when correlated to histopathology, where absolute measurements of features were obtained on data acquired on the day of animal sacrifice for histology. Features were selected as described and are presented in Table 2.

### Patient Data

3D DCE-US patient data was used as data for this exemplary embodiment to determine if treatment response could be predicted based on perfusion changes assessed with 3D DCE-US radiomics in patients with metastatic liver cancer receiving treatment. For this purpose, data from 7 adults were used. Inclusion criteria were: provide written consent, willing to comply with protocol, at least 18 years of age or older, and at least one liver metastasis from a gastrointestinal or pancreatic primary tumor confirmed with MRI or CT. A clinical oncologist referred each patient after introducing the study to the patient. Exclusion criteria were: documented anaphylactic or other severe reaction to any contrast media; pregnant or lactating patients; and patients with cardiac shunts or presence of severe pulmonary hypertension. Exclusions are based on contraindication for ultrasound contrast agent. No patient was excluded due to the exclusion criteria. Five patients were women with a mean age, 54.5 years; range, 48-60 years; 6 patients were men with a mean age, 57.6 years; range, 47-68 years. Included patients had liver metastases originating from the following primary tumors: rectal adenocarcinoma (n=2); pancreatic adenocarcinoma (n=1); pancreatic neuroendocrine tumor (n=4); and colonic adenocarcinoma (n=4). All 7 patients were scanned on Day 0 before initiating treatment, and on Day 14 after therapy start. At the end of the treatment cycle (around 60 days), treatment response was evaluated with an MRI/CT scan using “Response Evaluation Criteria in Solid Tumors (RECIST 1.1)” which is based on visible anatomical changes in size of the lesions. Briefly, the 3D DCE US scanned liver metastasis, among other tumor relevant lesions in the liver and/or other organs, was identified in the baseline CT scan before treatment start as “target lesion” and the size of all target lesions was again evaluated in the follow-up scan. The sum of diameter of all target lesions was assessed and treatment response classified as “Progressive Disease” (>+20% or new lesion), “Stable Disease” (<+20% to −30%), “Partial Response” (>−30%), or “Complete Response” (all target lesions disappeared). Based on that, patients with “Progressive Disease” were categorized in the current study as non-responder (n=3), and the rest as treatment responders (n=4).

TABLE 1
Summary of texture features

| Type | Features |
|---|---|
| Global texture features | Mean |
| (first-order gray-level statistics) | Median |
| | Mode |
| Standard first order | Standard Deviation |
| histogram-derived features. | Variance (1) |
| | Skewness |
| | Kurtosis |
| | Entropy (1) |
| Gray-Level Co-occurrence | Energy |
| Matrix (GLCM) | Contrast (1) |
| Most commonly used feature | Correlation |
| sets; uses a co-occurrence matrix | Homogeneity |
| looking at frequency of | Variance (2) |
| co-occurrence between two | Sum Average |
| pixels using a set | Entropy (2) |
| directionality and scale. | Dissimilarity |
| Gray-Level Run-Length Matrix | Short Run Emphasis (SRE) |
| (GLRLM) | Long Run Emphasis (LRE) |
| The matrix is based on the | Gray-Level Non-uniformity |
| run-length of voxels with | (GLN) (1) |
| same grey levels given a | Run-Length Non-uniformity (RLN) |
| specific direction. | Run Percentage (RP) |
| | Low Gray-Level Run Emphasis |
| | (LGRE) |
| | High Gray-Level Run Emphasis |
| | (HGRE) |
| | Short Run Low Gray-Level |
| | Emphasis (SRLGE) |
| | Short Run High Gray-Level |
| | Emphasis (SRHGE) |
| | Long Run Low Gray-Level |
| | Emphasis (LRLGE) |
| | Long Run High Gray-Level |
| | Emphasis (LRHGE) |
| | Gray-Level Variance (GLV) (1) |
| | Run-Length Variance (RLV) |
| Gray-Level Size Zone | Small Zone Emphasis (SZE) |
| Matrix (GLSZM) | Large Zone Emphasis (LZE) |
| This matrix is independent of | Gray-Level Non-uniformity |
| direction, and is built on the | (GLN) (2) |
| GLRLM methods as foundation. | |
| It estimates the size of zones | Zone-Size Non-uniformity |
| within an image that have the | (ZSN) |
| same grey levels. | Zone Percentage (ZP) |
| | Low Gray-Level Zone |
| | Emphasis (LGZE) |
| | High Gray-Level Zone |
| | Emphasis (HGZE) |
| | Small Zone Low Gray-Level |
| | Emphasis (SZLGE) |
| | Small Zone High Gray-Level |
| | Emphasis (SZHGE) |
| | Large Zone Low Gray-Level |
| | Emphasis (LZLGE) |
| | Large Zone High Gray-Level |
| | Emphasis (LZHGE) |
| | Gray-Level Variance (GLV) (2) |
| | Zone-Size Variance (ZSV) |
| Neighborhood Gray-Tone | Coarseness |
| Difference | Contrast (2) |
| Matrix (NGTDM) | Busyness |
| Aims to capture the grey scale | Complexity |
| difference between a pixel and | Strength |
| neighboring pixels. | |
| Features have been shown to | |
| capture true nature of texture in | |
| images similar to human | |
| perception. | |

TABLE 2
List of Selected Features: (Parametric Map)-(Feature)

| Selected Features | | Selected Features | Selected Features |
|---|---|---|---|
| (Length Scale = 0.3 mm) | | (Length Scale = 0.6 mm) | (Length Scale = 0.9 mm) |
| ′AIP-Energy′ | ′SIP-ZSV′ | ′AIP-RLV′ | ′AIP-Skewness′ |
| ′AIP-Variance (1)′ | ′SIP-LRE′ | ′AIP-Skewness′ | ′AIP-Kurtosis′ |
| ′AIP-LGZE′ | ′SIP-GLN (2)′ | ′AIP-Kurtosis′ | ′MIP-Sum Average′ |
| ′AIP-HGZE′ | ′SIP-HGRE′ | ′MIP-RLV′ | ′MIP-GLN (2)′ |
| ′AIP-SZLGE′ | ′SIP-SRHGE′ | ′MIP-Variance (2)′ | ′MIP-RLV′ |
| ′AIP-SZHGE′ | ′SIP-LRLGE′ | ′SIP-Variance (1)′ | ′MIP-Variance (2)′ |
| ′AIP-LZLGE′ | ′SIP-LRHGE′ | ′SIP-RLV′ | ′SIP-Contrast (1)′ |
| ′AIP-LRE′ | ′SIP-RLV′ | ′SIP-Variance (2)′ | ′SIP-Variance (1)′ |
| ′AIP-GLN (2)′ | ′SIP-Variance (2)′ | ′PE-Energy′ | ′SIP-SRHGE′ |
| ′AIP-LRLGE′ | ′PE-LRLGE′ | ′PE-Variance (1)′ | ′SIP-RLV′ |
| ′AIP-RLV′ | ′PE-RLV′ | ′PE-ZSV′ | ′SIP-Contrast (2)′ |
| ′AIP-Skewness′ | ′AUC-Energy′ | ′PE-GLN (2)′ | ′SIP-Variance (2)′ |
| ′AIP-Kurtosis′ | ′AUC-ZSN′ | ′AUC-Energy′ | ′SIP-Skewness′ |
| ′MIP-Variance (1)′ | ′AUC-SZHGE′ | ′AUC-SZE′ | ′AUC-Energy′ |
| ′MIP-LZLGE′ | ′AUC-LRE′ | ′AUC-ZSN′ | ′AUC-SZE′ |
| ′MIP-LRE′ | ′AUC-GLN (2)′ | ′AUC-LRE′ | ′AUC-LZE′ |
| ′MIP-GLN (2)′ | ′AUC-LGRE′ | ′AUC-GLN (2)′ | ′AUC-ZSN′ |
| ′MIP-HGRE′ | ′AUC-SRLGE′ | ′AUC-RLV′ | ′AUC-LZHGE′ |
| ′MIP-SRHGE′ | ′AUC-LRLGE′ | ′PE-Energy′ | |
| ′MIP-LRHGE′ | ′AUC-RLV′ | ′PE-Variance (1)′ | |
| ′MIP-Busyness′ | ′TP-ZP′ | | |
| ′MIP-Variance (2)′ | ′T0-LZLGE′ | | |
| ′SIP-Energy′ | | | |
| ′SIP-Variance (1)′ | | | |
| ′SIP-LZE′ | | | |
| ′SIP-GLN (1)′ | | | |
| ′SIP-HGZE′ | | | |
| ′SIP-SZHGE′ | | | |
| ′SIP-LZLGE′ | | | |
| ′AIP-Energy′ | | | |

(1). Fakih, Metastatic Colorectal Cancer: Current State and Future Directions, J. Clin. Oncol. 33, 1809-1824 (2015).
(2) Hurwitz, Bevacizumab plus irinotecan, fluorouracil, and leucovorin for metastatic colorectal cancer., N. Engl. J. Med. 350, 2335-2342 (2004).

### Results

### 3D Parametric Maps of Perfusion Parameters Display Longitudinal Perfusion Heterogeneity

A total of 78 mice bearing colon cancer tumors on the hind leg were used to identify multi-parametric biomarkers. Animals were either implanted with human LS174T or mouse CT26 colon cancer cells and were either left as control or treated with the anti-angiogenic agent Bevacizumab; only LS174T cells are responsive to Bevacizumab. The different cohorts allowed the inventors to develop a model based on image features (cohorts A and B), assess repeatability of image features (cohort C) and test the model in a separate data set with histology (cohort D). The processing pipeline as shown in FIG. 2 was used to extract 3D parametric maps of bolus-based tumor perfusion parameters from each data set on each imaging day. Briefly, this pipeline applies voxel-by-voxel fitting of a lognormal perfusion model to time-intensity curves (TIC) and generates a map for several bolus-based perfusion parameters (peak enhancement (PE), area under the curve (AUC), time to peak (TP), mean transit time (MTT) and bolus start time (TO)) using multi-core processing on a high-performance computing cluster. In addition to bolus-based perfusion parameters, 3 intensity projection volumetric images (maximum, average and standard deviation projection) were obtained from 4D data. Thus, for each 3D DCE-US data set, a total of 8 parametric maps and intensity projections (henceforth perfusion maps) were generated within a user-define volume of interest (VOI).

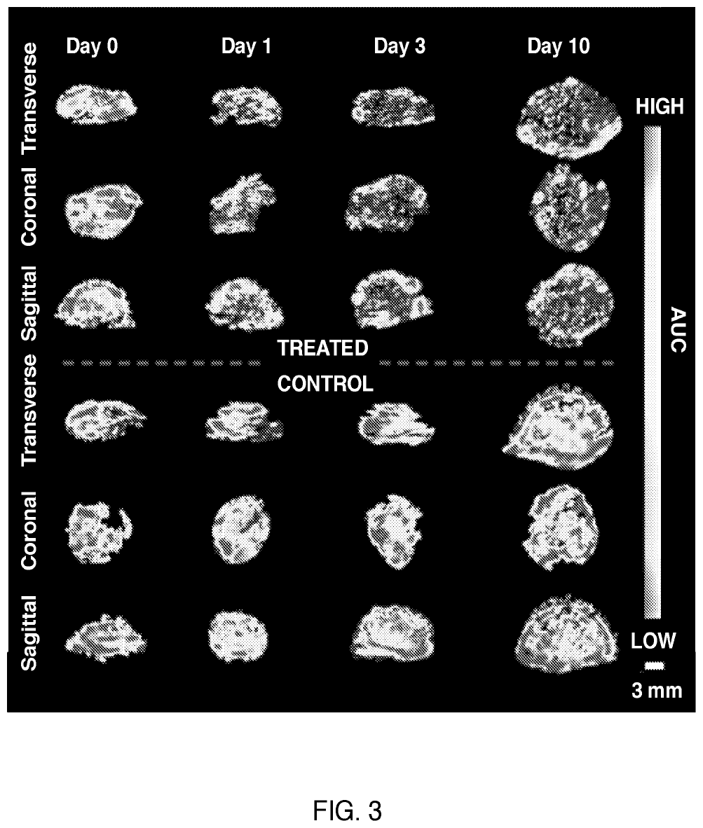

A representative perfusion map of the AUC is shown in FIG. 3. Note heterogeneous perfusion parameter values throughout the tumor tissue longitudinally. In both control and treated mice, tumors developed minimally perfused regions in the lesion by day 10, with persisting heterogeneous flow throughout the rest of the lesion. A qualitative decrease in the average AUC was noted throughout the whole lesion within 24 hours of the first dose of Bevacizumab in treated animals; overall lesion perfusion remained heterogeneous. Longitudinally, non-perfused (necrotic) regions developed in most control and treated tumors by days 7-10.

Features from Parametric Maps Improve Discrimination Between Tumor Groups

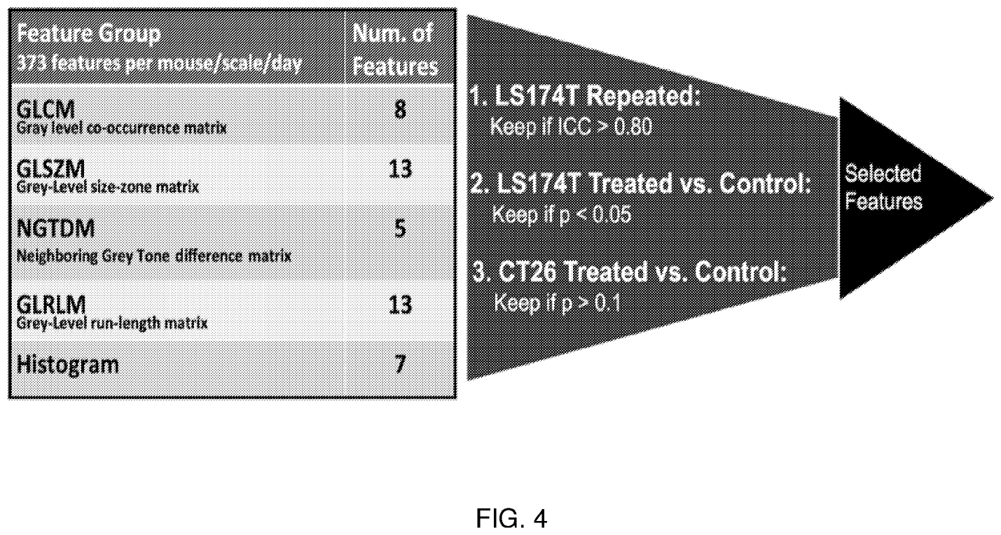

To compare image features extracted from 3D parametric maps to conventional ROI-averaged parameters, evaluated their performance in discriminating control and treated tumor groups exposed to Bevacizumab were both extracted both (FIG. 4). A total of 1128 quantified image features were extracted and evaluated per animal on each imaging day and calculated as percent difference from baseline (pre-treatment). The different treatment evaluation days allowed the inventors to evaluate different levels of treatment response relative to baseline. A complete list of all features is show in Table 1. The average TIC obtained from a VOI was also used to extract conventional quantitative bolus perfusion parameters (henceforth termed conventional parameters) using a log-normal fit to the ROI-averaged bolus curve. Conventional parameters are PE, AUC, TP and MTT; these were included in the complete set of 1128 features per animal/imaging day. To demonstrate that features from parametric maps can be sorted and combined to discriminate between tumor groups receiving Bevacizumab, we used two different methods; i) a linear statistical approach and, ii) a GLMNET approach.

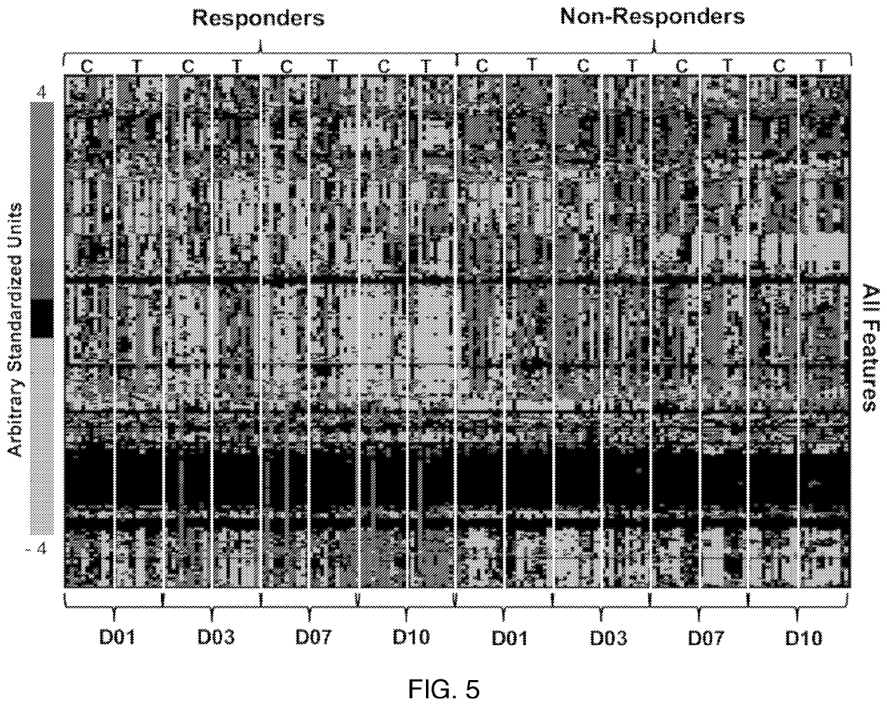

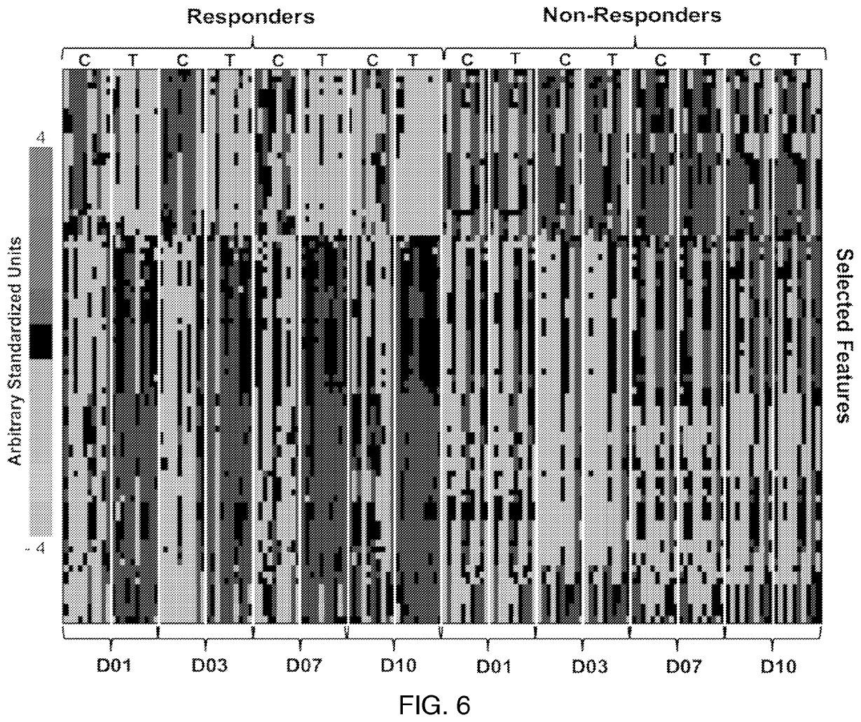

Statistical Approach—A statistics-based feature selection pipeline is presented in FIG. 4. This approach was chosen for its simplicity as a proof of concept. A heat map of all features is shown in FIG. 5, from which no distinct patterns can be seen between treated and control tumors in either the responder group (LS174T) or the non-responder group (CT26). Overall, a total of 90 features were selected that are presented as a heat map in FIG. 6 and in Table 2. Of the selected features, none were the conventional parameters PE, AUC, TP, or MTT. Note within the heat map (FIG. 6) the alternating pattern between treated (T) and control (C) animals for the responder group that is not observed in the non-responder group.

Principal component analysis (PCA) was used for dimensionality reduction to eliminate redundant and/or correlated features and identify the dominant dimensions, reducing the total number of features available. Ninety-eight percent of feature information was represented over 16 PCA components, while seventy percent of feature information was represented in 2 components. Overall, compared to conventional perfusion parameters (i.e. PE, AUC, TP, MTT), the two top dominant components performed better in discriminating between responders and non-responders on a subject-by-subject basis, with a Receiver Operator Curve-Area Under the Curve (ROC-AUC)>0.93 for each alone, in contrast to conventional parameters, which had a range of ROC-AUC of 0.40-0.75.

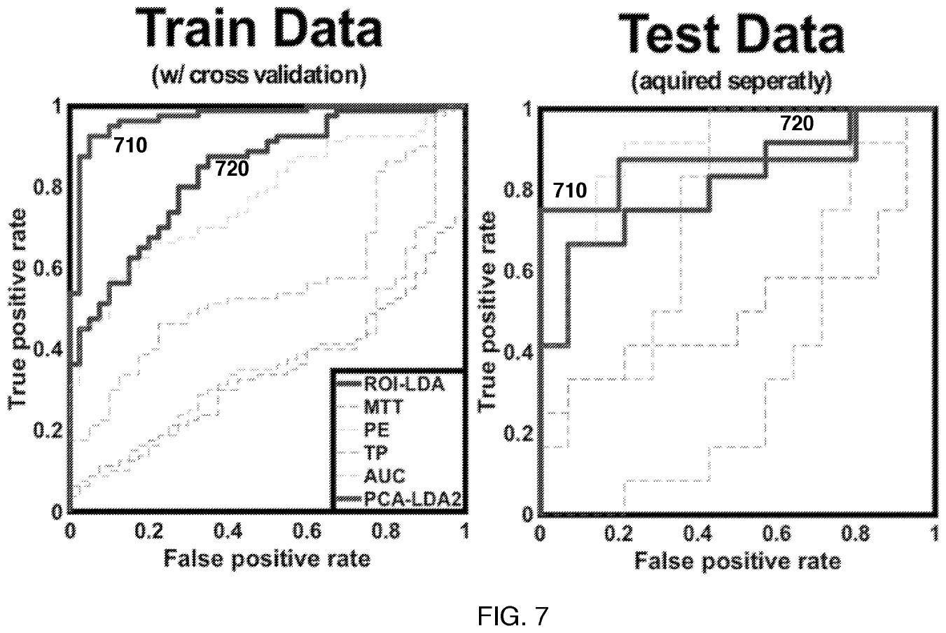

Two LDA models were generated based on PCA components, one was based on the top 16 PCA components (henceforth PCA-LDA1) and the other based on the top 2 PCA components (henceforth PCA-LDA2); these were generated using data from all of Cohort A/B over all treatment days and tested with 10-fold cross-validation and separately on Cohort D. Similarly, an LDA model based on the four main conventional ROI-averaged parameters (PE, AUC, TP, MTT) combined (henceforth ROI-LDA) was generated using the same data set and tested on Cohort D. ROC curves for the PCA-LDA2 and ROI-LDA models, along with ROC curves for each individual conventional parameter in Cohort A/B (Train with cross-validation) and Cohort D (Separately acquired test data), are shown in FIG. 7. While the ROI-LDA model had a ROC-AUC of 0.78 for Cohort A/B and 0.66 for the test Cohort D, the PCA-LDA2 model had a ROC-AUC of 0.96 for Cohort A/B (Train with cross-validation) and 0.88 for the test Cohort D (Separately acquired test data). PCA-LDA1 model also performed well, with a ROC-AUC of 0.97 for Cohort A/B and 1 for the test Cohort D. All ROC-AUC are summarized in Table 3.

TABLE 3
Area under curve (AUC) for ROC analysis to discriminate
between responders and non-responders. CI is 95% confidence interval.

| | Data Set A/B | Data Set D | Patients |
|---|---|---|---|
| | (Train Data) | (Test Data) | (Test Data) |
| GLMNET | — | 0.95 | 0.95 |
| | | (CI: 0.83, 1.00) | (CI: 0.83, 1.00) |
| PCA-LDA1 | 0.97 (CI: 0.93, | 1 (CI: 1.00, | 1 (CI: 1.00, 1.00) |
| | 1.00) | 1.00) | |
| PCA-LDA2 | 0.96 (CI: 0.91, | 0.88 | 0.93 (CI: 0.69, 1.00) |
| | 1.00) | (CI: 0.77, 1.00) | |
| ROI-LDA | 0.78 (CI: 0.68, | 0.66 | 0.50 (CI: 0.04, 0.96) |
| (Conventional) | 0.87) | (CI: 0.40, 0.92) | |
| PE-LDA | 0.74 (CI: 0.64, | 0.76 | 0.66 (CI: 0.22, 1.00) |
| | 0.83) | (CI: 0.53, 0.99) | |
| AUC-LDA | 0.43 (CI: 0.33, | 0.51 | 0.42 |
| | 0.52) | (CI: 0.23, 0.79) | (CI: −0.03, 0.87) |
| MTT-LDA | 0.62 (CI: 0.51, | 0.65 | 0.25 |
| | 0.72) | (CI: 0.39, 0.91) | (CI: −0.13, 0.63) |

GLMNET Approach—A GLMNET-based approach was also tested to differentiate between responders and non-responders. This approach was chosen due to its ability to handle high-dimensionality data. GLMNET uses a mixture of L2 and L1 regularization in fitting a generalized linear model, and as such, will “select features” by setting coefficients to 0 via the Lasso L1 component. Training of the GLMNET was done with Cohort A/B. All features were fed into the model as is to distinguish between responders and non-responders and to allow the model to select features using the complexity penalty method. Consistent results were achieved across all tested alpha values, with ROC-AUC of 0.95 for mouse test data (Cohort D) and average ROC-AUC of 0.95 for human test data, indicating a well generalizing model.

### Biomarkers are Correlated to Histological Quantification of Vascular Density

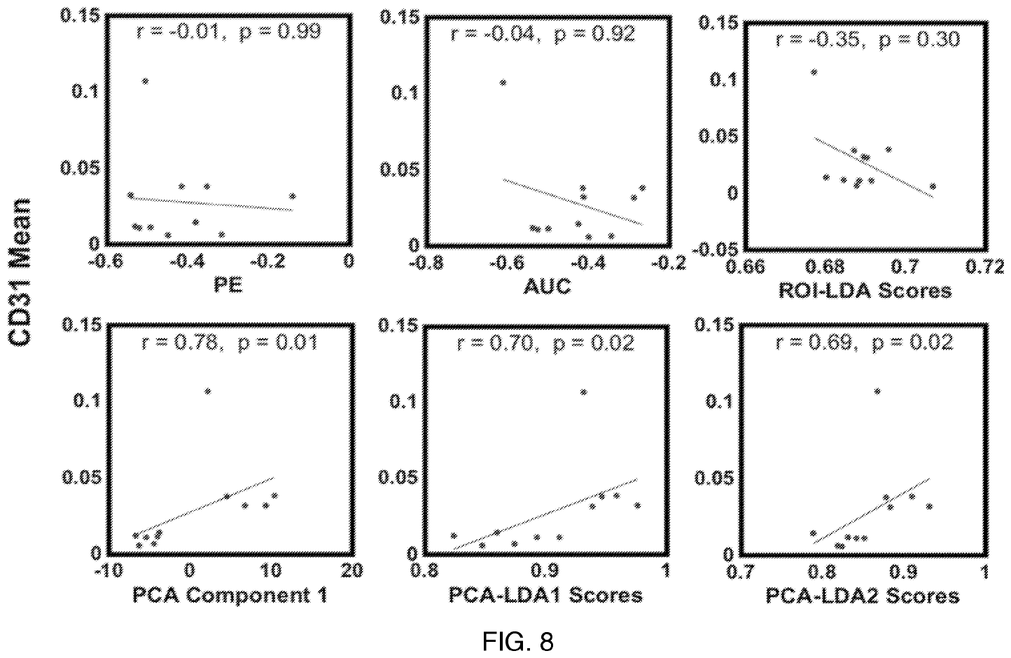

The inventors tested whether selected PCA components and the scores from the PCA-LDA model from the statistical approach were correlated to volumetric histological characterization of mean vascular density (MVD) on CD31-stained tissue slides of the tumors. Parametric map image features were taken as absolute measurements to compare directly to histological measures, as opposed to change relative to baseline as per above, and were tested for correlation against volumetric histological evaluation of MVD at day 1 (24 hours after therapy) in Cohort D. The Spearman correlation coefficient was obtained between the MVD and each of the 16 PCA components and was found to correlate significantly (p<0.05) to 10/16 components. The first component had the best correlation coefficient R (R=0.78, p=0.01), and is presented in FIG. 8. The scores from the PCA-LDA1 and PCA-LDA2 models correlated with histology with an R of 0.70 and 0.69 (P=0.02), respectively (FIG. 8 bottom row). In contrast, none of the conventional parameters alone, or combined in the ROI-LDA scores, correlated with histology (FIG. 8 top row).

### Multi-Parametric Models Based on Features are Predictive of Early Patient Treatment Response

Human 3D DCE-US longitudinal bolus data acquired over 2 weeks was obtained (Day 0 before treatment and Day 14 after treatment start) in patients receiving cancer therapy. All longitudinal data had a 60-day RECIST 1.1-based response evaluation as reference standard; responders were those reported with stable or regressed disease, and non-responders were those with progressive disease based on RECIST 1.1. All characteristics pertaining to acquired clinical data are summarized supra and Table 4.

TABLE 4
Longitudinal Patient Data

| Primary Disease | Treatment | Response | Size | Age | Sex |
|---|---|---|---|---|---|
| Colorectal | FOLFOX + | Regression | 9 cm | 60 | M |
| Adenocarcinoma | Bevacizumab | | | | |
| Pancreatic | Temozolomide + | Stable | 2.4 cm | 61 | M |
| Neuroendocrine | Capecitabine | | | | |
| Pancreatic | Capecitabine + | Stable | 1.6 cm | 54 | M |
| Adenocarcinoma | Oxaliplatin | | | | |
| Colorectal | Irinotecan + | Regression | 4.3 cm | 52 | M |
| Adenocarcinoma | Bevacizumab | | | | |
| Pancreatic | Capecitabine + | Progression | 2.6 cm | 70 | F |
| Adenocarcinoma | Oxaliplatin | | | | |
| Colorectal | RRx-001 | Progression | 2.2 cm | 68 | M |
| Adenocarcinoma | | | | | |
| Pancreatic | Everolimus + | Progression | 9.5 cm | 52 | M |
| Neuroendocrine | Octreotide + | | | | |
| | Embolization | | | | |

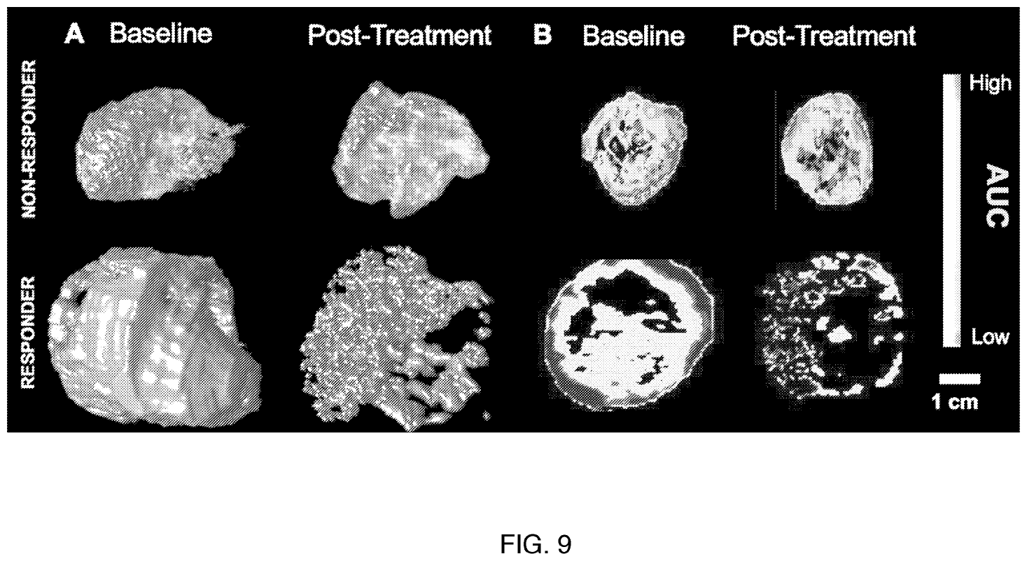

For each data set, eight parametric maps were generated using the same methods as described supra, and image features were extracted for each parametric map to feed into the mouse-trained PCA-LDA1, PCA-LDA2, ROI-LDA and GLMNET models. Parametric maps for three representative patients are shown in FIG. 9. In the context of all patients, perfect discrimination was observed for PCA-LDA1 (AUC of ROC=1). Similarly, a ROC-AUC of 0.92 for PCA-LDA2 was noted. In contrast, a ROC-AUC of 0.33 for an LDA with all the conventional parameters was observed. For the GLMNET approach, the human data was used as test data with a non-responder/responder split of 3:4, indicating a well-balanced test set. With a model trained purely on mouse data, a human test set was able to achieve an AUC of 0.95.

### CONCLUSIONS

The potential of 3D DCE-US radiomics to predict early treatment response using quantitative image features extracted from bolus-based perfusion maps is demonstrated. The results indicate that image features can yield repeatable multi-parametric biomarkers that are better than conventional parameters at discriminating between pre-clinical responders and non-responders in training and test data and that can be directly translated to the clinic to differentiate between human responders and non-responders within 14 days of treatment based on tumor perfusion changes. The data also demonstrates that multi-parametric biomarkers are significantly correlated to CD31 MVD from histology (p=0.02-0.01). In summary, volumetric maps of perfusion parameters reflect the heterogeneity of perfusion following therapy and image features capture heterogeneous perfusion changes.

The method of this invention statistically quantifies patterns of perfusion through interconnected voxel patterns, as opposed to the intensity of contrast which can vary with the injection of the bolus and the number of microbubbles injected. Thus, DCE-US perfusion maps potentially offer more repeatable quantification.

The results demonstrate that histogram and texture features from volumetric parametric maps maximize perfusion information beyond conventional averaged parameters and are better at differentiating between responders and non-responders. The inventors were surprised to find high correlations for both individual PCA components and PCA-LDA1 scores to volumetric CD31 MVDs, especially given that conventional bolus parameters have been previously reported to not correlate with histology. This supports that patterns captured through features are more representative of heterogeneous tumor perfusion than conventional parameters. Another important aspect of this invention is the use of pre-clinical data to identify perfusion features that change in treated animals known to respond to treatment. Using these features, the inventors developed models that capture tissue perfusion changes, which we found to translate to the clinic in pilot human data.

The results set the stage for more precise quantification of perfusion from 3D DCE-US for treatment monitoring using a radiomics approach, before anatomical changes are overtly visible based on current Response Evaluation Criteria in Solid Tumors (RECIST 1.1). Based on this invention work, further development of machine-learning models to predict responders based on improved feature-based quantification of perfusion patterns in volumetric data is promising. Beyond this, the invention demonstrates that early perfusion changes measured using image features in tumor tissues are predictive of treatment response. The bedside availability of 3D DCE-US can thus positively impact health care costs and provide rapid decision support in managing cancer patient treatment regimens.

Embodiments of the invention are envisioned as a method or system, a computer-implemented method or processing pipeline executable by a computer or computerized system, platform or chip either as a stand-alone device or in conjunction with an imaging or ultrasound system.

What is claimed is:

1. A method for quantitative tissue characterization, classification and discrimination to capture different patterns of tissue perfusions, comprising:
(a) having two or three-dimensional dynamic contrast enhanced ultrasound (DCE US) data of a contrast bolus perfused tissue;
(b) generating parametric perfusion maps of contrast bolus tissue perfusion parameters representing the DCE US data;
(c) extracting statistical parameters for each of the generated parametric perfusion maps, wherein the statistical parameters are first order statistical parameters, second order statistical parameters, or a combination thereof, wherein the extracting statistical parameters are based on underlying perfusion characteristics; and
(d) classifying, discriminating, or a combination thereof the perfusion maps of the tissue using the extracted statistical parameters.

2. The method as set forth in claim 1, wherein the statistical parameters are histogram features, texture or radiomic features, or a combination thereof.

3. The method as set forth in claim 1, wherein the parametric perfusion maps represent a temporal behavior of the contrast within a pixel mapped over a two-dimensional space or a three-dimensional space.

4. The method as set forth in claim 1, wherein the parametric perfusion maps represent a temporal behavior of the contrast of a single pixel or a window of a group of pixels mapped over a two-dimensional space or a three-dimensional space.

5. The method as set forth in claim 1, wherein the second order statistical parameters summarize interconnectivity of voxels.

6. The method as set forth in claim 1, wherein the second order statistical parameters are obtained for different image resolution scales, different imaging pixel or voxel angles, or a combination thereof.

7. The method as set forth in claim 1, wherein the second order statistical parameters are based on the interconnected nature of pixels or voxels in the parametric perfusion maps.

8. The method as set forth in claim 1, wherein the second order statistical parameters capture a statistical relationship of one voxel to another voxel with the aim of capturing intensity patterns and heterogeneities with quantified values from different parametric perfusion maps.

9. The method as set forth in claim 1, generating multi-parametric features by reducing the dimensionality of the number of correlated statistical features.

10. The method as set forth in claim 1, further comprising using statistical or machine learning to characterize tissue, classify a disease or a combination thereof.

11. The method as set forth in claim 1, further comprising using supervised machine learning, unsupervised machine learning or deep learning approaches to classify tissues.

12. The method as set forth in claim 1, further comprising applying an image pre-processing method, a segmentation method, a pixel intensity binning method, or a combination thereof to enhance the generation of the parametric perfusion maps or the statistical characterization of the parametric perfusion maps.
